(19) United States
(12) Patent Application Publication
(10) Pub. No.: US 2011/0118546 A1
(43) Pub. Date: May 19, 2011

(54) BALLOON CATHETER WITH DETACHABLE HUB, AND METHODS FOR SAME

(75) Inventors: Travis E. Dillon, Winston-Salem, NC (US); Andres F. Aguirre, Chicago, IL (US); Kevin Chmura, Lewisville, NC (US)

(73) Assignee: Cook Endoscopy/Wilson Cook, Winston-Salem, NC (US)

(21) Appl. No.: 12/909,285

(22) Filed: Oct. 21, 2010

Related U.S. Application Data
(60) Provisional application No. 61/256,773, filed on Oct. 30, 2009.
(60) Provisional application No. 61/256,755, filed on Oct. 30, 2009.
(60) Provisional application No. 61/329,243, filed on Apr. 29, 2010.

(51) Int. Cl. A61B 1/00; A61B 1/018

(52) U.S. Cl. 600/106; 600/116; 600/115

(57) ABSTRACT

A balloon catheter may include a distal anchoring balloon and a proximal hub that is removable from the catheter body. The catheter body may include a valve structure providing for maintaining the balloon in an inflated state during and after removal of the proximal hub. The valve preferably is constructed such that removal of the hub provides a low-profile proximal catheter end that will allow that proximal catheter end to pass through an endoscopic surgical device such as, for example, through an accessory channel of a standard duodenoscope and/or an ultra-slim endoscope/cholangioscope, facilitating a scope-exchange for use during, for example, a cholangioscopy or pancreatoscopy procedure. A method useful for scope exchange and/or introducing another elongate surgical device may utilize a balloon catheter with a distal anchoring balloon and a proximal hub that is removable from the catheter body.

### CROSS-REFERENCE TO RELATED APPLICATIONS

This application claims priority to U.S. Provisional Application Ser. Nos. 61/256,773 and 61/256,755, both filed Oct. 30, 2009, and 61/329,243, filed Apr. 29, 2010, each of which is incorporated herein by reference in its entirety.

### TECHNICAL FIELD

Embodiments of the claimed invention relate to a medical balloon catheter device configured for passage through an ultra-slim endoscope. More particularly, embodiments of the claimed invention relate to a balloon catheter including a proximal actuatable catheter lumen seal and a detachable hub, and methods of use.

### BACKGROUND

Intraductal endoscopes have an increasingly important role in the diagnosis and nonsurgical treatment of biliary and pancreatic diseases. Early attempts to inspect the biliary and pancreatic ducts endoscopically have been hampered by technical limitations of the scopes. More recently, the development of fine-caliber flexible scopes known as fiber optic miniscopes has obviated many of these problems and has provided a valuable new tool for a growing number of indications. These miniature endoscopes can be used intraoperatively, during endoscopic retrograde cholangiopancreatography (ERCP, commonly performed peroral), and percutaneous transhepatic cholangiography (PTC).

Peroral cholangioscopy is usually performed by two experienced endoscopists using a “mother-baby” scope system, in which a thin fiberscope is inserted into the working channel of a large therapeutic endoscope (e.g., a duodenoscope). Smaller and more durable miniscopes allow for an accessory channel of their own. This accessory channel of the miniscopes permits sampling for histological and cytological examination and the insertion of catheters for dye or probes for laser or lithotripsy. Miniscopes such as cholangioscopes can also be used for pancreatoscopy.

The mother-baby scope technique can be expensive with regard to personnel and equipment: two endoscopists plus assistants, two image processors (one for each camera), expensive fiber optics in the baby scope that can often be damaged during standard manipulation with resulting image degradation, etc. The standard 1.2 mm working channel of fiber optic baby scopes limits diagnostic and therapeutic options. It is therefore desirable to provide an endoscope configured to function as a cholangioscope by being dimensioned to be navigable through hepatic and pancreatic ducts. Such scopes are currently available, but they encounter problems of efficient introduction to a patient's biliary duct in a procedure that provides high quality images (e.g., superior to fiber optics imaging) at a desirable procedure cost. These problems include the difficulty (or impossibility) of navigating a larger fiber optic baby scope having a greater than 1.2 mm working channel through a mother scope (e.g., duodenoscope), out its side-facing distal accessory channel end past and manipulated by the elevator, and then into a patient's biliary duct. If one is to introduce a small scope (along the size of a “baby scope” or smaller) into the biliary ducts or other patient body structure without a primary (e.g., “mother”) scope, it is necessary to provide some type of “navigating track” because the smaller scopes are not sufficiently rigid/robust to be directed/navigated independently and directly through the esophagus, stomach, and duodenum to, for example, the common biliary duct.

Accordingly, techniques are being developed to conduct direct peroral cholangioscopy (POC). Direct POC requires only a single endoscopist working with a single image processor, using a CMOS or CCD (rather than—and with image quality superior to—fiber optic) camera system that provides a 2 mm (rather than 1.2 mm) accessory channel and that can be used with existing scopes, image processors, and monitors. One example of such improved technology is disclosed in “Overtube-balloon-assisted direct peroral cholangioscopy by using an ultra-slim upper endoscope” (Choi, et al.; Gastrointestinal Endoscopy, 69(4):935-40; April 2009), where an over-tube with a balloon of the type used for double-balloon enteroscopy was directed into the duodenum adjacent the Ampulla of Vater with an ultra-slim scope supported in the lumen of the over-tube, whereafter the scope was directed into the previously-dilated bile duct.

It would be advantageous to provide materials for efficient introduction of an ultra-slim scope suitable for cholangioscopy and pancreatoscopy in conjunction with use of a standard-sized endoscope (e.g., duodenoscope) that can be exchanged out without significant loss of procedural efficiency, but without limiting the equipment and/or procedure to a mother-baby scope configuration, and also providing for easier, more efficient navigation into the bile duct or other locations.

### BRIEF SUMMARY

In certain embodiments, aspects of the present invention may include a balloon catheter device including a removable hub and configured to function as an anchored guide for an endoscopic surgical device such as an endoscope or other endoscopic surgical device.

### BRIEF DESCRIPTION OF THE DRAWINGS

FIGS. 1A-1H show a cholangioscopy and biopsy procedure including a scope exchange using an anchoring balloon catheter with a removable hub;

FIG. 2 is an example of a catheter hub;

FIG. 3 is another example of a catheter hub, embodied as a manifold;

FIG. 4A shows a balloon catheter embodiment;

FIGS. 4B-4D show detail views of portions of the balloon catheter embodiment of FIG. 4A;

FIGS. 4E and 4F show the balloon catheter of FIG. 4A with the proximal catheter-sealing valve actuated, and a detail of one valve embodiment, respectively;

FIG. 4G shows a step of removing the manifold from the catheter body of the balloon catheter embodiment of FIG. 4A;

FIGS. 5A-5C show another balloon catheter embodiment with a catheter-sealing valve and removable hub;

FIGS. 6A-6C show longitudinal section and exterior views of another balloon catheter embodiment with a side-aperture valve;

FIG. 6D shows an alternative embodiment of the balloon catheter of FIGS. 6A-6C;

FIGS. 7A-7D show an embodiment of a balloon catheter including a grooved piston valve embodiment;

FIGS. 7E and 7F show two embodiments for connecting the housing to the catheter body of the embodiment shown in FIGS. 7A-7D;

FIG. 8 shows another balloon catheter embodiment, with an elongate wire valve;

FIGS. 9-9D show another balloon catheter embodiment, with a distal flap-type valve;

FIG. 10 shows another embodiment of a catheter device with a removable manifold; and

FIGS. 10A-10B show a partial longitudinal section view of the device 1000 of FIG. 10.

### DETAILED DESCRIPTION

### Definitions

Ultra-slim endoscopes, as that term is used herein, refer to endoscopes having an outer diameter of about 6.0 mm or less (including less than 5.0 mm). The term “hub” refers to the proximal end structure of a balloon catheter including a connection structure (e.g., Luer-type or other fluid-patent connection) configured for effective connection to provide a path of fluid communication between a source of inflation fluid, a catheter inflation lumen, and a balloon lumen, and includes manifold-style hubs that may have more complex or ancillary structures. The terms “distal” and “proximal” are to be understood with their standard usages, referring to the direction away from and the direction toward the handle/user end of a tool or device, respectively (i.e., away from and toward the patient, respectively).

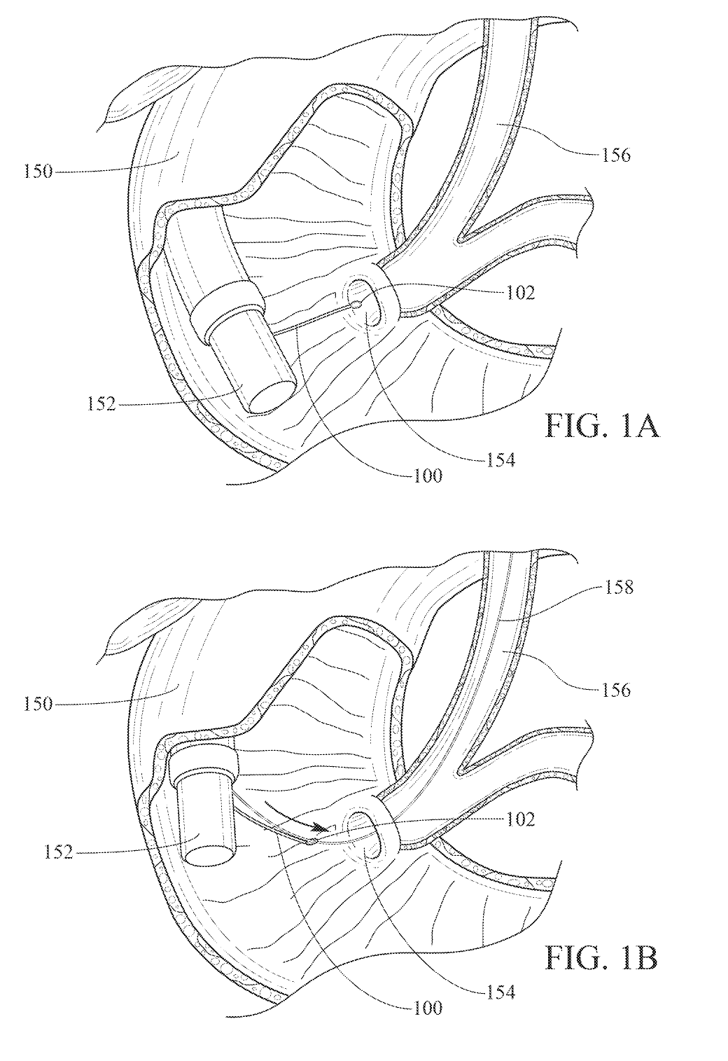

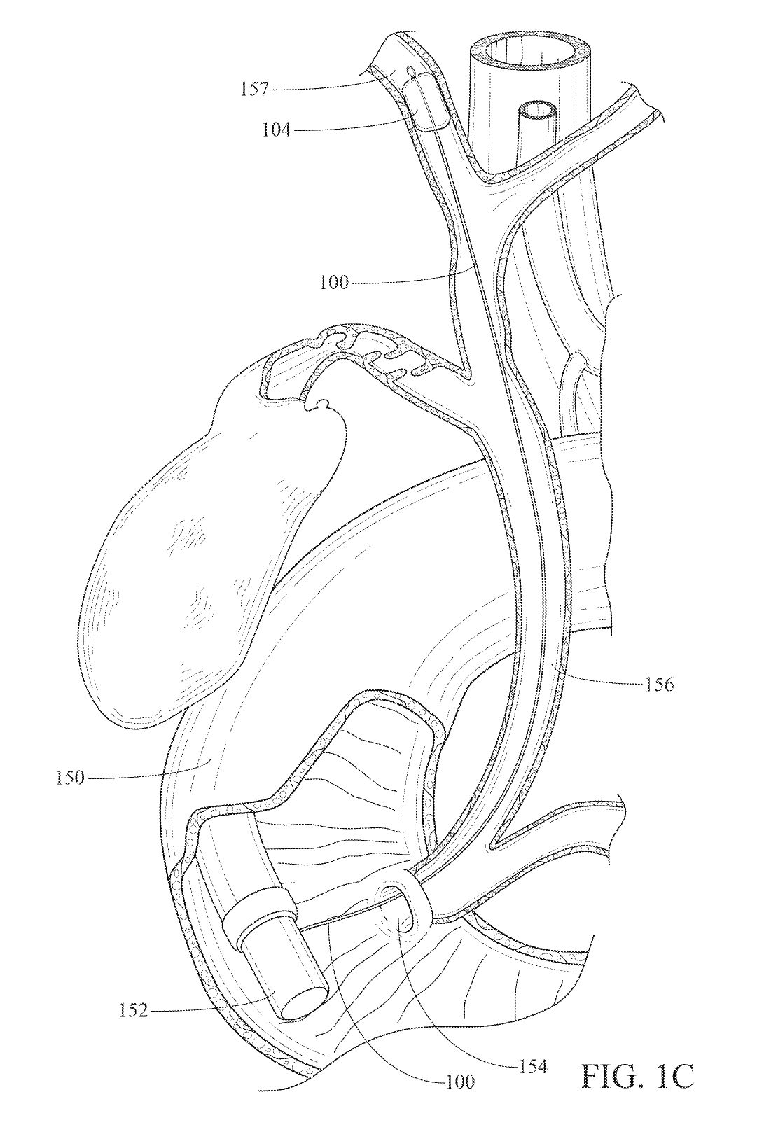

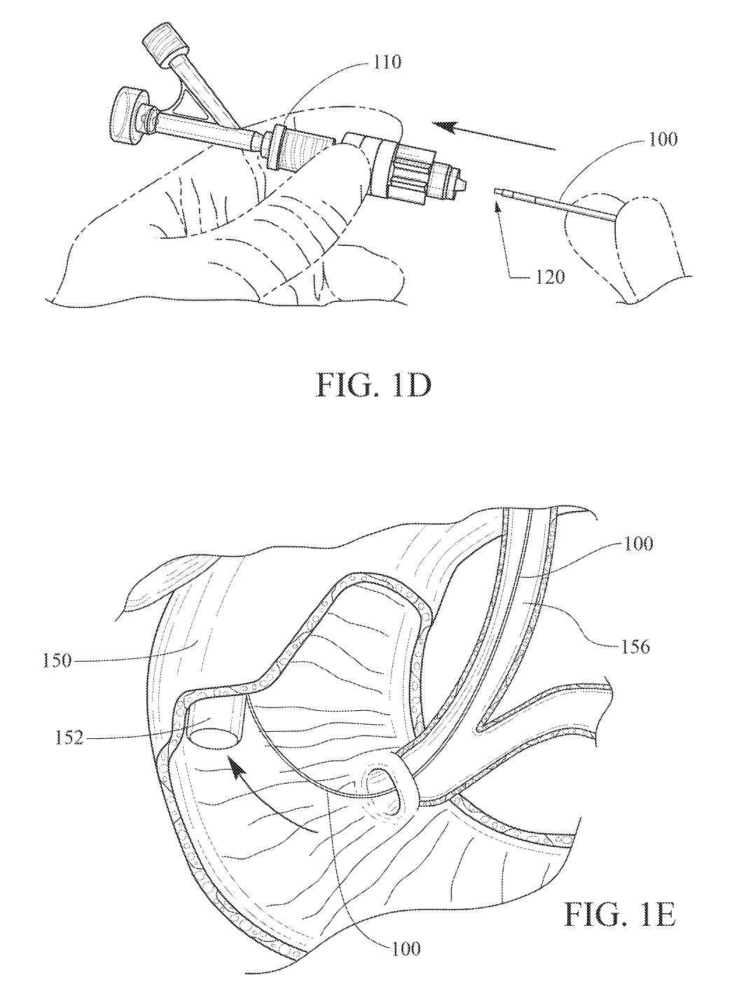

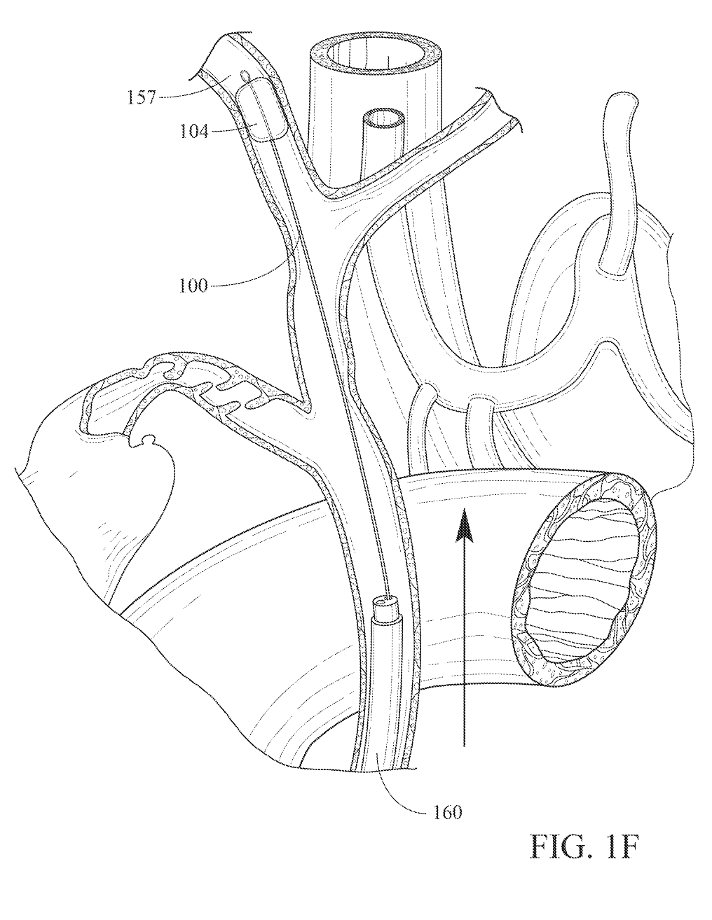

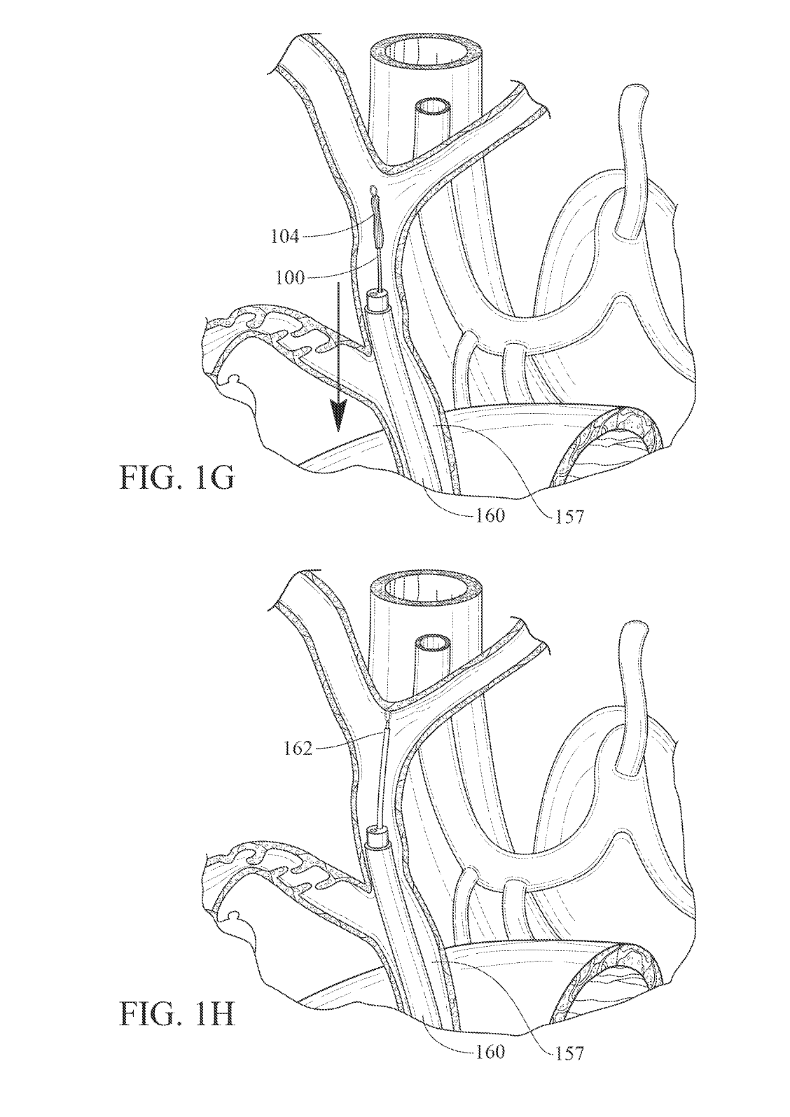

A cholangioscopy procedure using a scope-exchange facilitated by a balloon catheter including a proximal actuatable sealing valve and removable hub is described with reference to FIGS. 1A-1H. Embodiments of different catheters including a proximal actuatable catheter-sealing valve and removable hub are described thereafter.

FIG. 1A shows a side-viewing endoscope embodied as a duodenoscope 152 that has been directed into the duodenum 150 of a patient adjacent the Ampulla of Vater about the Sphincter of Oddi 154, which is shown as having been cannulated (e.g., through a sphincterotomy). A loop-tipped catheter 100 extending through a working channel of the duodenoscope 152 is shown being directed through the cannulated sphincter 154 into the common bile duct 156.

FIG. 1B shows an alternative method for introducing the loop-tipped catheter 100 through the cannulated sphincter 154 into the common bile duct 156 using a wire guide 158. In this embodiment, the wire guide 158 is first navigated into the common bile duct 156. Then, the loop 102 of the catheter 100 is looped around the wire guide 158 and directed in monorail fashion therealong into the common bile duct 156.

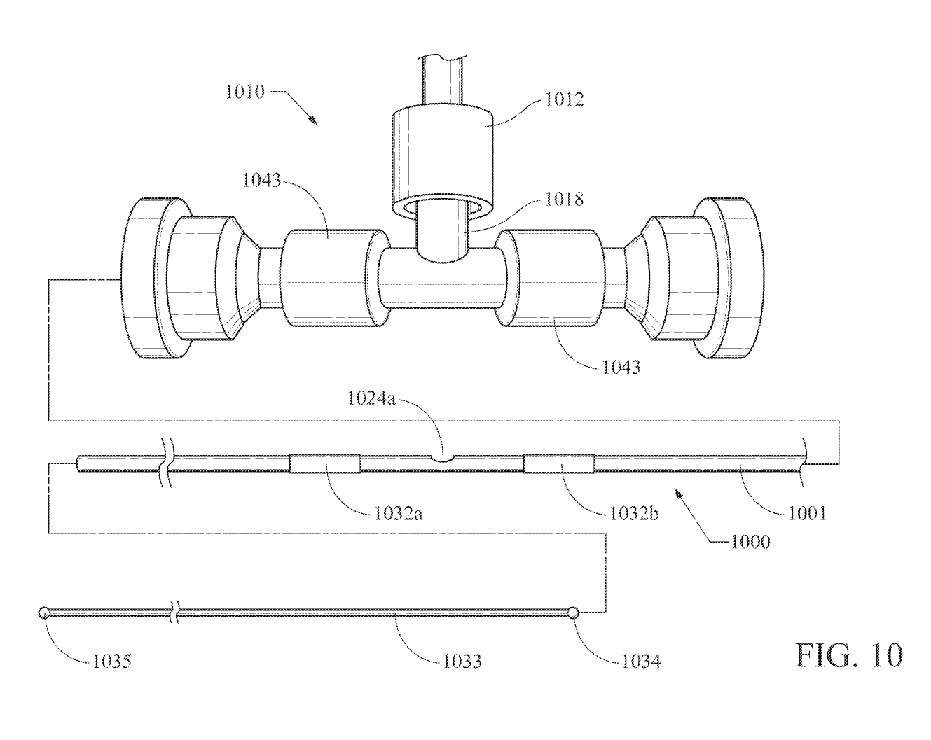

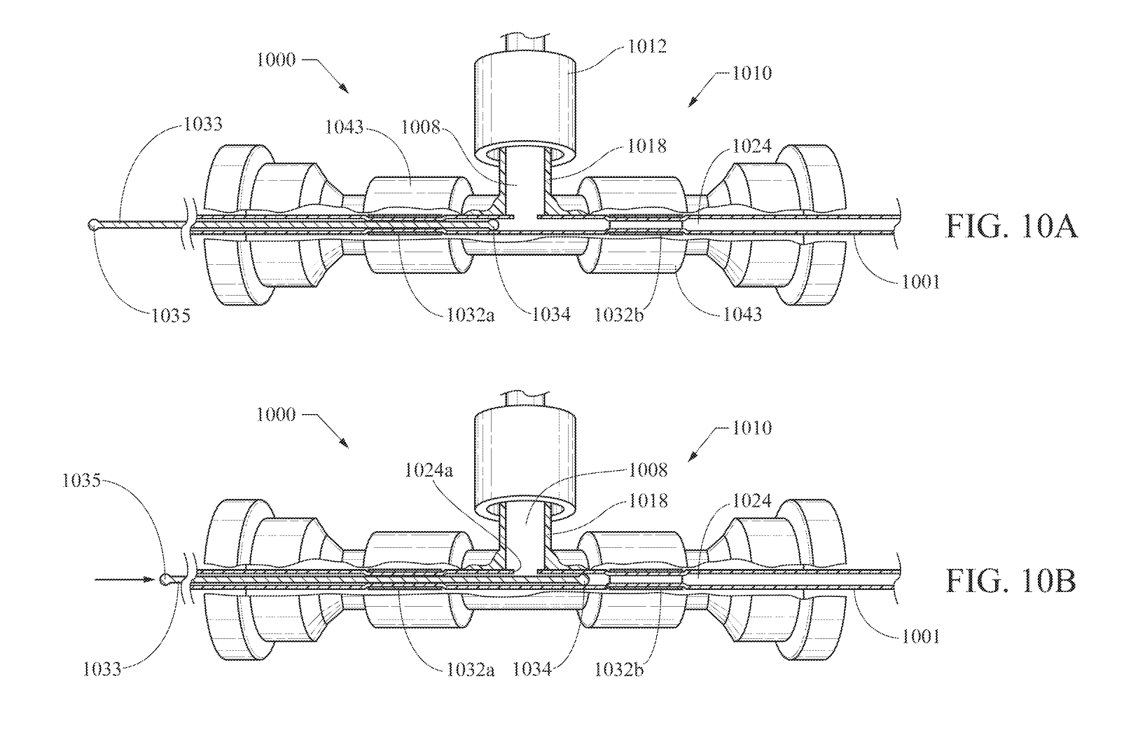

Regardless of which method is used to direct the catheter 100 into the common bile duct 156, the catheter 100 may be directed further into the hepatic branch side (or pancreatic duct side) of the common bile duct 156. Then, as shown in FIG. 10, the balloon 104, which preferably will be a compliant balloon, may be inflated (e.g., as shown in the hepatic duct 157, although it may be anchored in the common bile duct 156 or a different branch, including in the pancreatic duct as those of skill in the art will appreciate that pancreatoscopy may also be practiced within the scope of the present invention). It is preferable that the balloon 104 be inflated sufficiently to anchor the catheter 100, but that it does not significantly distend the ductal surface contacted by the inflated exterior balloon surface. Compliant balloons may be made of latex or other biocompatible material having desirable elasticity. In some embodiments, a balloon may be non-compliant in accords with desirable manipulation during a surgical procedure.

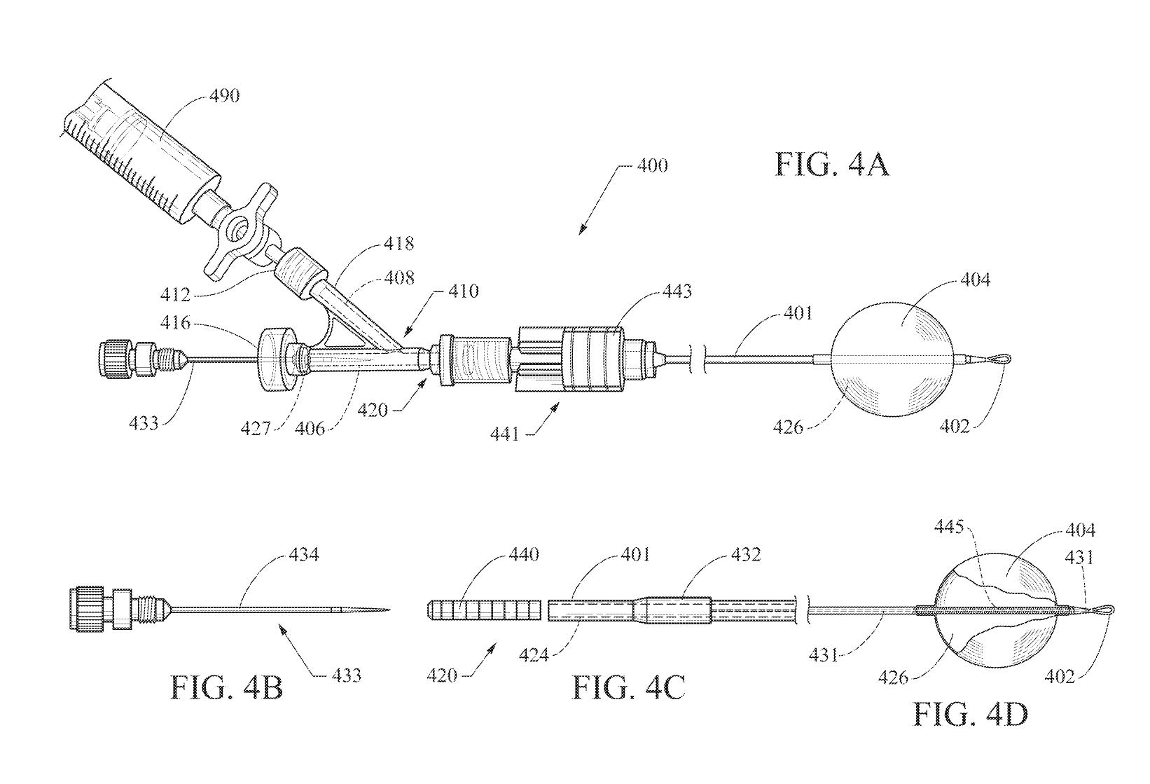

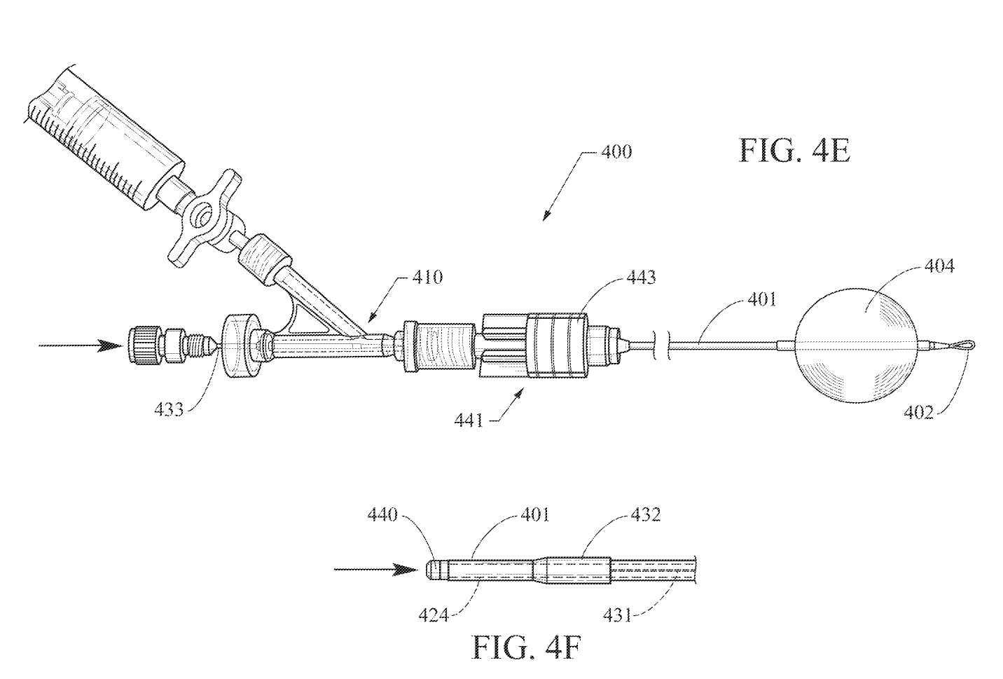

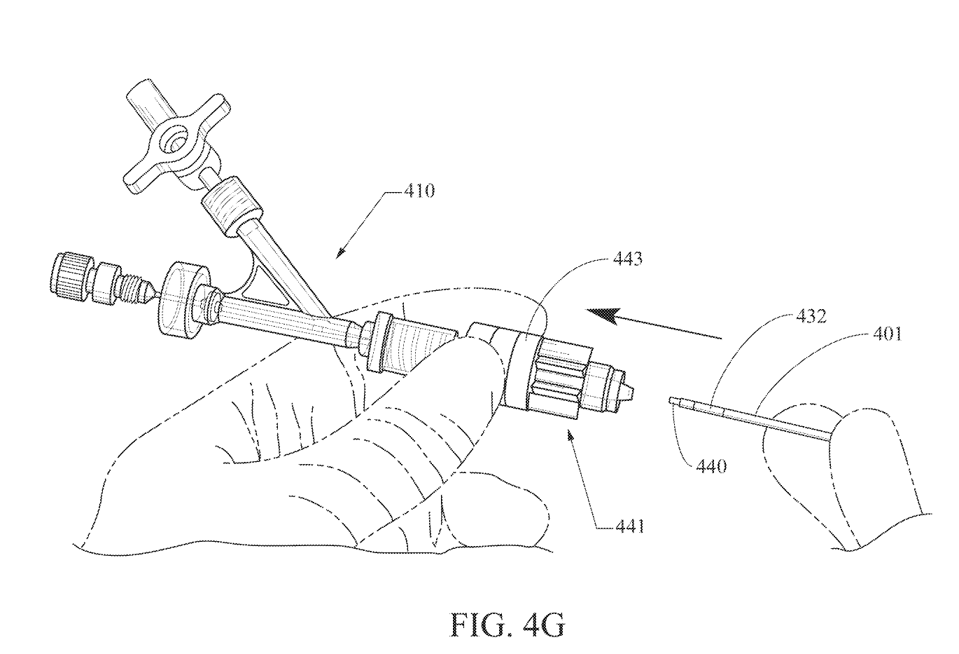

FIG. 1D shows the proximal end of the balloon catheter 100, with a hub embodied as a manifold 110 being detached therefrom. Prior to detachment of the manifold 110, a valve 120 is actuated to seal the proximal end of the balloon catheter 100 to maintain inflation fluid pressure in the balloon 104 (in the present application, an “actuated valve” is in a closed configuration, and a “non-actuated valve” is in an open configuration). The valve and manifold may be embodied in the manner described with reference to FIGS. 4A-8 below, with features combined therefrom, or with another valve/sealing structure covered by the claims including all equivalents. As will be appreciated with reference to FIG. 1E, this removal of the proximal manifold 110 allows a user to withdraw the duodenoscope 152 over the catheter 100 while the catheter 100 remains in place, anchored by the balloon 104 (as shown in FIG. 1C).

Next, an ultra-slim endoscope 160 is directed distally along the catheter 100. Specifically, the proximal catheter end is inserted into the distal end of an accessory/working channel of the ultra-slim scope 160. Then, as shown in FIG. 1F, the catheter 100 may serve as a guide, allowing the distal end of the ultra-slim scope 160 to be directed into the common bile duct 156. Thereafter, as shown in FIG. 1G, the balloon 104 may be deflated (e.g., by allowing the inflation fluid to escape or by providing negative pressure to withdraw it using a syringe or vacuum source) and the catheter 100 withdrawn, freeing up the accessory channel of the ultra-slim scope 160. A user may then introduce a diagnostic or therapeutic instrument through the accessory channel of the ultra-slim scope 160 such as, for example, biopsy forceps 162 as shown in FIG. 1H.

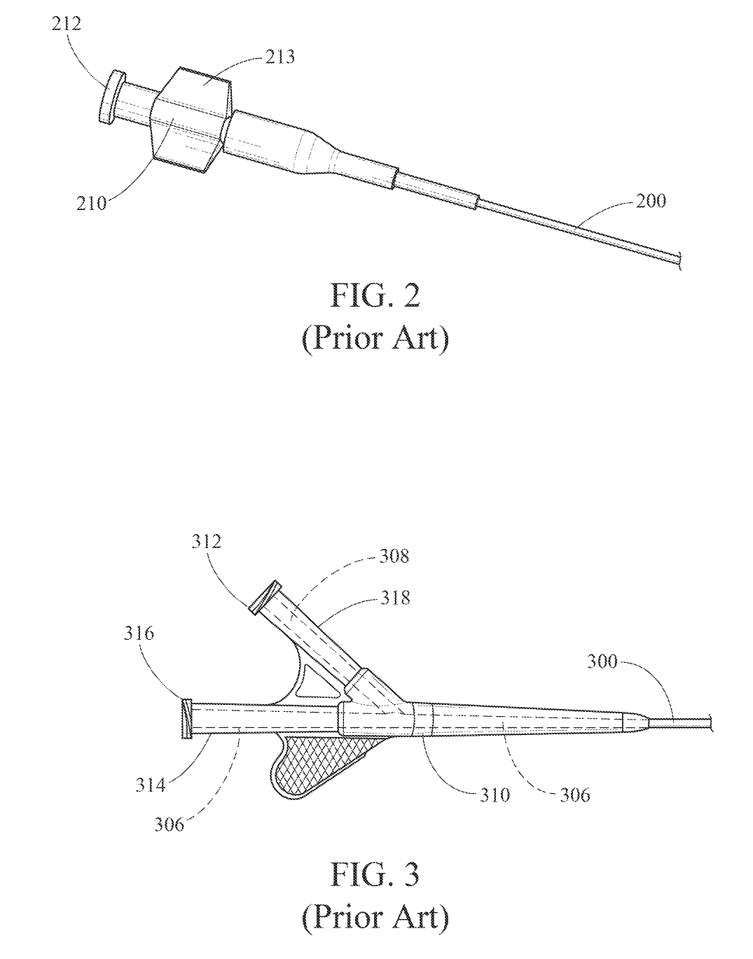

FIG. 2 shows a conventional basic catheter hub 210 for a catheter 200. The hub 210 includes a Luer-type connector 212 and wings 213 configured to facilitate manipulation. FIG. 3 shows a conventional catheter hub configured as a manifold 310. The manifold 310 includes a Luer-type connector 312 on a side branch 318 and another connector 316 on a linear branch 314 that is substantially coaxial with the longitudinal axis of the catheter 300. The manifold 310 includes a main lumen 306 that is in fluid communication with a lumen 308 of the side branch 318. Such conventional hubs, including manifolds, are fixedly and irremovably attached to the catheter body. It will be appreciated that the outer diameter and/or cross-sectional area of these and other conventional hubs are such that they would not fit through an elongate surgical device such as, for example, a lumen of a large-bore catheter, polymer biliary stent, working/accessory channel of an endoscope or other minimally-invasive image-capture device.

Embodiments of the presently-disclosed device and method include a hub that is removable from a catheter body, including a sealing structure such as a valve that is configured to maintain inflation fluid/pressure in a balloon sufficient to keep that balloon and catheter anchored in a duct of a patient body while an elongate surgical device is passed over a proximal end of the catheter (with the hub removed). Alternatively, or in addition, a hub may be reattached to aid in deflating the balloon. Valve embodiments of the present invention preferably provide a transverse cross-sectional area that is less than or at least not substantially greater than the transverse cross-sectional area of the catheter. With this configuration, an elongate surgical device (e.g., duodenoscope, ultra-slim endoscope, other camera or image-capturing device, polymer stent, larger-bore catheter, etc.) may be passed over the entire length of a catheter device (including the valve) of the present invention when the balloon is deflated, and/or the entire length of the catheter device may be passed through a central lumen, working channel, or other opening of the elongate surgical device. In other words, the outer diameter of the valve and of the balloon when deflated most preferably is not significantly greater than the outer diameter of the elongate catheter body, such that the entire device (with the hub removed) may be passed through the lumen of an elongate surgical device.

FIGS. 4A-4D show, respectively, a balloon catheter 400 with a removable hub embodied as a manifold 410 (FIG. 4A) and detail illustrations of a seal-actuation stylet 433, plug-style valve 420, and a longitudinal section view of the distal catheter end with balloon 404 and loop-tip 402. As shown in FIG. 4A, the manifold 410 includes an inflation syringe 490 attached to its side branch 418 at a connector end 412. A branch lumen 408 provides a path of fluid communication from the syringe 490 to the main lumen 406, which is in fluid communication with the inflation lumen 424 of the elongate catheter body 401 and, thereby, the balloon inflation lumen 426. A proximal portion of the main manifold lumen 406 includes a Tuohy-Borst seal 427 that provides for passage therethrough of the seal-actuation stylet 433 without significant loss of inflation fluid pressure from the inflation lumens 424, 426. The phrase “Tuohy-Borst seal” is intended to include the specific structure associated in the art with that name, as well as all equivalent simple seals configured for maintaining fluid-patency during introduction of a solid item through a seal.

The manifold 410 is attached to the elongate body 401 of the catheter 400 by a fluid-tight compression seal 441 including a sliding member 443 that enforces a compression fit when in the distal position shown, and that releases the catheter body when retracted proximally. Other connectors suitable for fluid-tight but detachable connection of a manifold to a catheter body (e.g., threaded, bayonet-connector, gasket/friction-fit) are known or may be developed in the future and practiced within the scope of the present invention. The balloon 404 is shown as inflated.

The seal-actuation stylet 433 is shown in the detail view of FIG. 4B. It includes a metal or other generally rigid distal body 434 and a proximal structure 435 configured for engaging/disengaging the seal-actuation stylet 433 with the proximal main body connector 416 of the manifold and for longitudinal manipulation of the body 434 within the main manifold lumen 406.

A proximal end of the catheter body 401 (generally obscured by the manifold 410 in FIG. 4A) is shown in the detail view of FIG. 4C. The catheter 400 includes a stiffening wire 431 embedded in its wall some distance distal of the absolute proximal catheter end. A cannula 432 bridges the “wired” and “non-wired” catheter region. A simple valve 420 includes the proximal end of the catheter 400 and a plug 440. The plug 440 is shown as slightly proximal of the absolute proximal catheter end, such that the valve 420 is in an open/non-actuated state that will allow free passage of an inflation fluid through the catheter inflation lumen 424.

FIG. 4D shows a partial longitudinal section view of the distal portion of the catheter assembly of FIG. 4A. The balloon 404 is shown around the distal body portion of the catheter 400. A generally helical metal coil 445 may be disposed in the catheter in this distal portion to provide structural strength for navigating the catheter 400 and to reinforce the catheter body in a region where one or more apertures (not shown) are included to provide a path of fluid communication from the catheter lumen 424 into the balloon lumen. The loop-tip 402 is attached to the stiffening wire 431, and—in the illustrated embodiment—is sealed with the catheter 400 by a generally frustoconical adhesive or polymer structure that also seals the distal end of the catheter inflation lumen. The loop-tip 402 preferably provides a generally atraumatic distal end that will facilitate navigation through body lumens and also permit monorail-style navigation along a wire guide as described above with reference to FIG. 1B.

Actuation of the valve 420 and removal of the manifold 410 from the catheter 400 are described with reference to FIGS. 4E-4G. FIGS. 4E and 4G show a user having advanced the seal-actuation stylet 423 distally against the plug 440. FIG. 4F shows that this action actuates the valve 420 by engaging the plug 440 into the proximal end of the catheter inflation lumen 424, which will maintain the pressure needed to keep the balloon 404 inflated as shown in FIG. 4E by occupying and substantially sealing the catheter inflation lumen 424. FIG. 4G shows the manifold 410 with the compression seal 441 having been disengaged by retracting the sliding member 443 proximally. This disengagement releases the sealed proximal end of the catheter body 401, allowing an elongate surgical device (e.g., duodenoscope, ultra-slim endoscope, polymer stent, larger-bore catheter) to be moved over that end during or after a scope exchange or similar scope manipulation as is described above with reference to FIGS. 1A-1H. The plug 440 may be manually removed from the proximal catheter end to allow deflation of the balloon 404.

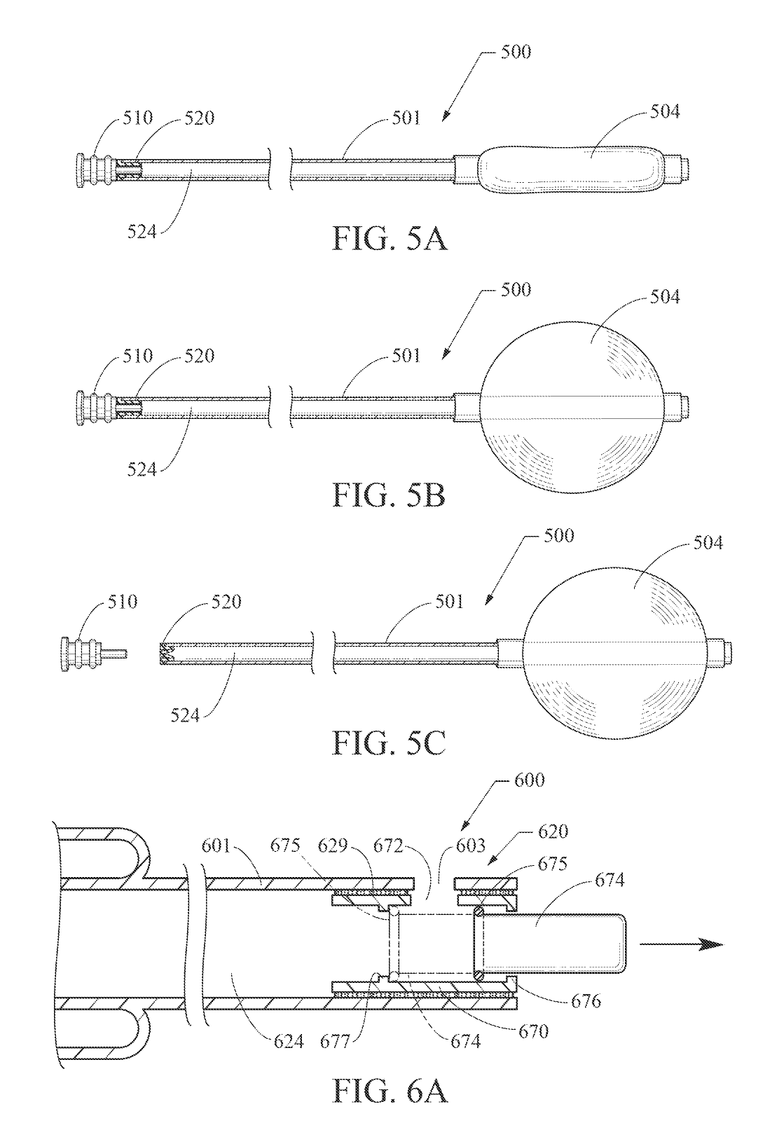

FIGS. 5A-5C show a partial longitudinal section view of another embodiment of a balloon catheter 500 including an elongate catheter body 501, a removable hub 510, and a method of use thereof. FIG. 5A shows the catheter 500 with a balloon 504 in a deflated state. The proximal hub 510 is shown as a very basic hub, but may alternatively be embodied as a hub like the ones shown in FIGS. 2-3 or other hubs (including manifolds) now known or later developed. An actuatable valve is embodied as a pliable seal 520 configured to substantially form a seal sufficient to retain inflation fluid in the catheter inflation lumen 524 when/where the seal contacts itself. The seal 520 is configured to seal around the distal end of the hub 510 as shown in FIG. 5A. FIG. 5B shows the balloon 504 inflated, with the hub 510 still in place through the seal 520.

FIG. 5C shows the seal 520 in an actuated state, effected by proximal retraction and removal of the hub 510 therefrom. Removal of the hub 510 allows the pliable surface of the seal 520 to collapse and contact itself in a sealing manner that will maintain sufficient inflation fluid pressure in the balloon lumen and catheter inflation lumen 524. The seal 520 may be constructed of an elastic material such as latex, silicone (including a gel-filled and/or intact-gel silicone construction), soft acrylic polymer or any material similar to any of these in structure and/or function, provided said material will effect a suitable seal in the circumstances described. In contrast to other embodiments shown herein, which may require a separate actuation step, the valve seal 520 is self-actuating, that is it is actuated automatically by the act of removing the hub 510. Other valve embodiments may be modified within the scope of the present invention to obtain the same function. This and other embodiments preferably are configured to allow reattachment of the hub 510 in a manner that will re-open the valve seal 520 and facilitate deflation of the balloon 504.

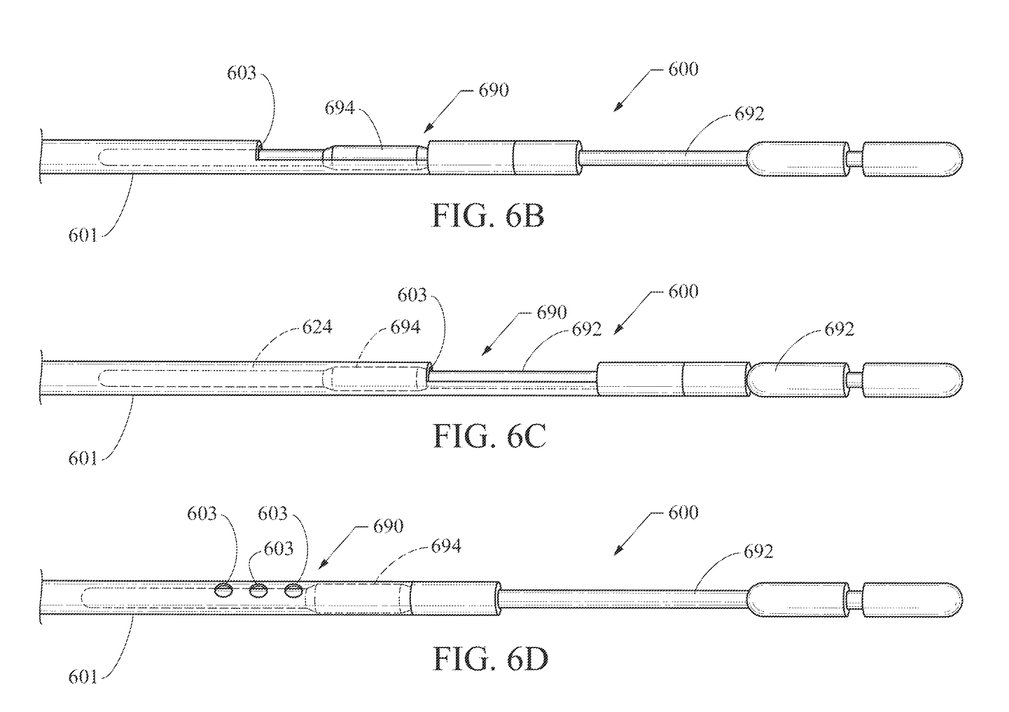

FIGS. 6A-6D show proximal portion views of other embodiments of a catheter 600 with a proximal actuatable valve 620 and a distal balloon 604. This valve 620 may be configured for use with a removable hub or manifold 410 such as the one shown in FIGS. 4A, 4E, and 4G and referred to by reference here, which reference will readily be understood by those of skill in the art (e.g., by envisioning insertion of the catheter 600 into a manifold as described). In the present embodiment, the catheter 600 includes a side aperture 603 configured to align with the branch lumen 408 of the manifold 410 when the manifold is attached to the catheter body 601. In this manner, the branch lumen 408 will provide a path of fluid communication with the catheter lumen 624.

In the embodiment shown in FIG. 6A, the valve 620 includes a generally cylindrical housing 670 retained by overmolding, friction fit, or adhesive 629 within the proximal end of the catheter lumen 624. The housing 670 includes at least one side aperture 672 configured to at least partially align with the catheter side aperture 603 to provide a path of fluid communication with the catheter lumen 624. The inner diameter of the housing 670 includes a proximal stop 676 and a distal stop 677. The valve 620 also includes a generally columnar plunger 674 with a flared distal end 675 disposed slidably between the proximal and distal stops 676, 677. The flared distal end 675 may be a continuous structure with the plunger 674, or it may be formed as an o-ring set into a groove or other inset at or near the distal end of the plunger 674.

As shown in FIG. 6A, the plunger 674 drawn in solid-line is in a proximal position with its flared distal end 675 disposed near or against the proximal stop 676. This position will not significantly occlude the housing aperture 672 or the catheter aperture 603, thereby providing a free, patent path of fluid communication between the manifold branch lumen 408 and the catheter lumen 624. FIG. 6A also shows a dashed-line image of the plunger 674 in a valve-actuated configuration where the flared distal end 675 is disposed near or against the distal stop 677, substantially forming a seal preferably sufficient to retain inflation fluid in the balloon 604 and catheter lumen 624. Actuation of the valve 620 in conjunction with use of a hub like the manifold 410 may be effected in the same manner as actuating the plug 440 of FIG. 4F—by using a stylet (e.g., stylet 423) to push the plunger 674 distally into the actuated position, thereby sealing the catheter lumen 624 to allow removal of the hub 410 without significant loss of inflation fluid or balloon volume. When desirable, the plunger 674 may be retracted again to allow for deflation of the balloon 604.

FIGS. 6B-6C show an alternative embodiment of the catheter 600 including a valve 690 without an internal housing. The valve 690 includes at least one side aperture 603 and a plunger 692 with an end portion 694 dimensioned to fully occupy a cross-sectional area of the catheter lumen 624. The plunger 692 is shown in FIG. 6B as being located proximal of the distal end of the aperture 603 such that inflation fluid may freely flow through the aperture 603 into/out of the catheter lumen 624. The valve 690 may be actuated/closed by distal advancement of the plunger 692 such that the plunger end portion 694 will fully occlude the catheter lumen 624, creating a seal that will allow removal of a hub without deflating a distal balloon attached thereto. The embodiment shown in FIG. 6D is substantially similar to that shown in FIGS. 6B-6C, except that its side aperture is embodied as a plurality of side apertures 603 that may selectively be blocked or left open by the end portion 694 of the plunger 692.

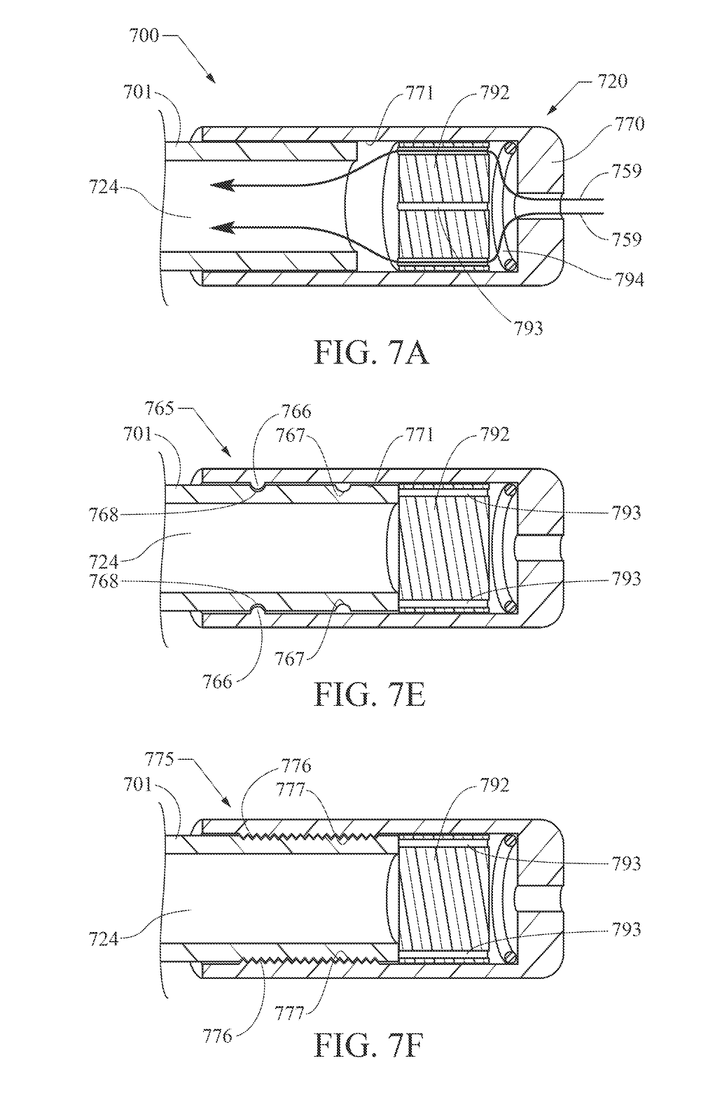

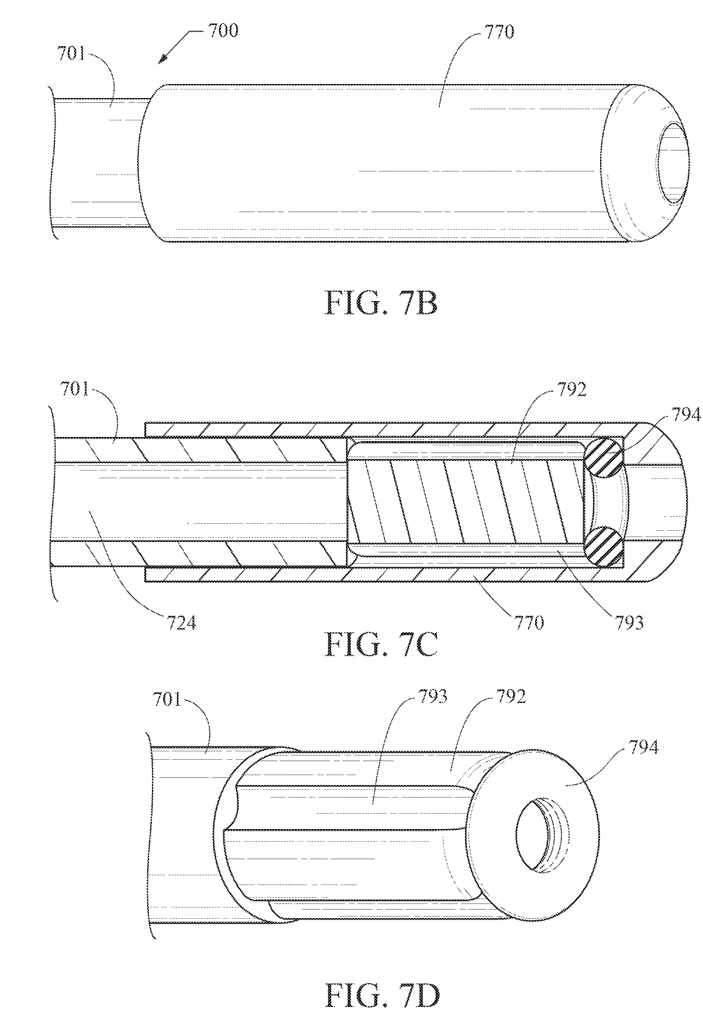

FIGS. 7A-7F show another valve embodiment 720 for a balloon catheter 700 including an elongate catheter body 701 and a detachable hub (not shown). The valve 720 includes an outer housing 770 with an inward-facing surface 771 that may be longitudinally-movably secured to the catheter body 700 with, for example, a detent connection 765 (described below with reference to FIG. 7E), a threaded connection 775 (described below with reference to FIG. 7F), or other connection mechanism providing for controlled longitudinal movement of the housing relative to the catheter body 701. The catheter body 701 and housing 770 are generally shown in longitudinal section. A grooved piston 792 is longitudinally slidably disposed within the housing 770 and preferably is dimensioned to contact or very nearly contact the inner diameter of the housing. At least at its distal end, the depth of its grooves 793 is equal to or less than a thickness of the wall of the catheter body 701. An o-ring 794 may be disposed at the proximal end of the piston 792 within the housing.

FIG. 7A shows a longitudinal section view of the valve 720 in a non-actuated/open position, with arrow-tipped lines 759 indicating the path of fluid communication for inflation fluid through the proximal end of the housing 770, along the grooves 793, and into the catheter lumen 724. FIG. 7B shows an exterior view of the housing 770 and body 701 of the catheter 700. FIG. 7C shows a longitudinal section view of the valve 720 in an actuated/closed position, wherein the housing 770 is distally advanced onto and relative to the catheter 700. Within the housing 770, the catheter 700 generally seals the distal ends of the grooves 793 and the o-ring 794 substantially forms a seal between the proximal ends of the grooves 793 and a proximal inner face of the housing 770. FIG. 7D shows an end perspective view of the valve position shown in FIG. 7C, illustrating the relative positions of the catheter 700, grooved piston 792, and o-ring 794 as they would appear in a closed/sealed configuration with the housing 770 removed.

The housing 770 may be attached to the catheter 700 by frictional contact between generally smooth surfaces as shown in FIGS. 7A and 7C. However, it may be preferably to provide a more secure engagement. FIG. 7E shows a detent connection 765 between the inward-facing surface 771 of the housing 770 and the outer surface of the catheter 700. When the valve is non-actuated (in an open/free-flow configuration), a first circumferential detent ridge 766 on the inward-facing surface 771 of the housing 770 will substantially sealingly engage a first circumferential groove 767 on the catheter exterior surface. As shown in FIG. 7E, when the valve 720 is actuated (in an closed configuration), the first circumferential detent ridge 766 on the inward-facing surface 771 of the housing 770 substantially sealingly engages a second circumferential groove 768 on the catheter exterior surface. In the embodiment shown in FIG. 7F, the inner surface of the distal housing portion includes a threaded surface 776 that mates with a complementarily threaded exterior surface 777 of the catheter body 701. It will readily be appreciated how this valve embodiment may be sealed by advancingly engaging the threaded surfaces 776, 777 to draw and seal the piston 792 and housing 771 firmly against the catheter body 710.

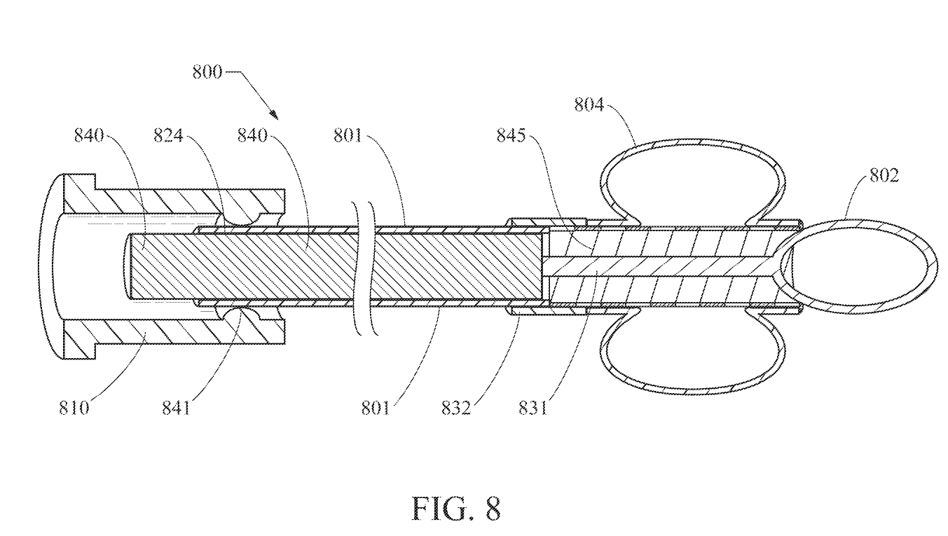

FIG. 8 shows another embodiment of a balloon catheter 800 with a hub 810 detachably connected to a tubular body 801. The removable proximal hub 810 is attached to the tubular body 801 in this embodiment by a friction fitting 841. Tubular body 801 includes a longitudinal lumen 824 extending therethrough and providing a path of fluid communication with a distal balloon 804. The tubular body 801 includes a distal metal coil 845 configured for providing structural support of the distal end including a loop tip 802, which is connected to a longitudinal stiffening wire 831 embedded in the wall of the tubular body 801. A cannula 832 may be included to provide structural reinforcement across the end of the core wire 840 and the portion of the tube body 801 supported only by the coil 845 and stiffening wire 831.

A valve/seal allowing removal of the hub 810 for a scope-exchange or other action without losing inflation pressure of the balloon 804 is provided by an elongate flexible solid core wire 840 that generally (but not completely) occupies a cross-sectional area of the tube lumen 824. In preferred embodiments, the outer diameter of the tube body 801 will be dimensioned to allow easy passage over its outer surface of an ultra-slim endoscope. In addition, it is preferable that it include externally and internally lubricious surfaces to allow movement of the core wire 840 and overlying structures without damaging or significantly moving the tube body 801 if/when it is anchored in a patient's body structure by its balloon 804. The very close tolerance of the core wire outer diameter and tube inner diameter will form an effective seal, minimizing or stopping loss of inflation fluid from the balloon 804 when inflated to anchor the device 800 during a procedure (e.g., as shown in FIGS. 1A-1H), but inflation can be effected using a high-pressure fluid-introduction source configured to overcome the flow resistance of the close tolerance. The core wire 840 may be removed from the tube lumen 824 to allow deflation of the balloon 804.

In one exemplary embodiment, the tube 801 may be constructed of PEEK with a silicone coating, having an outer diameter of about 0.035 inches (about 0.89 mm) and an inner diameter of about 0.023 inches (about 0.58 mm), with a core wire constructed of nitinol and having an outer diameter of about 0.021 to about 0.0215 inches (about 0.53 to about 0.55 mm), with a gold coil lip tip and platinum-gold coil-spring base, and a female Luer hub.

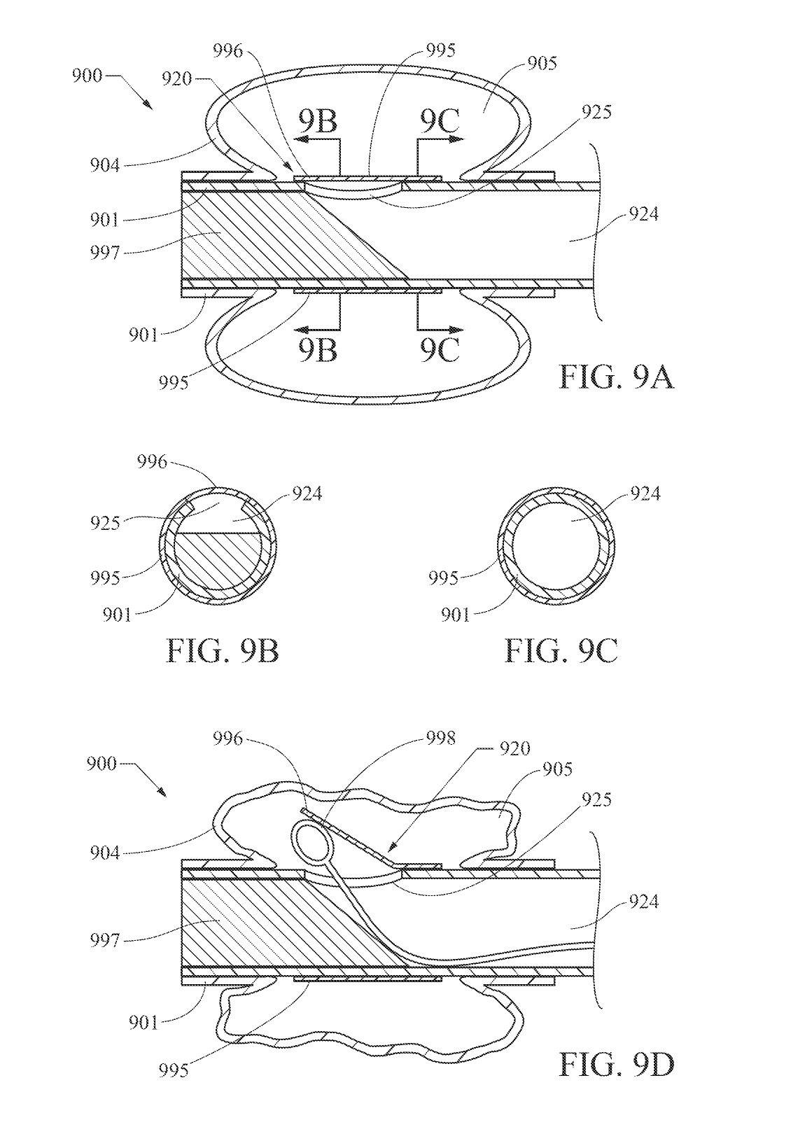

A distal portion of another balloon catheter 900 with a detachable hub (not shown) is shown in partial longitudinal section in FIGS. 9A-9D. It includes an elongate catheter body 901 having a catheter lumen 924 and a balloon 904. An aperture 925 provides a path for fluid communication between the balloon lumen 905 and the catheter lumen 924. A valve mechanism 920 includes a valve sleeve 995 disposed around the catheter wall 901, providing a fluid-tight seal. The valve sleeve 995 includes a valve flap 996 that is shown covering the aperture 925 in a fluid-tight manner in FIG. 9A, where pressure from inflation fluid in the balloon lumen 905 keeps the flap 996 sealed against the catheter wall 901 when the balloon is inflated, but which may be opened to allow inflation by distal pressure of inflation fluid being introduced through the catheter lumen. The surfaces of the flap and/or catheter wall where they contact may be treated (e.g., with tacky adhesive, gel material, static charge, magnetic materials) to an enhanced but disruptable fluid-tight contact therebetween. A ramp 997 occupies and seals the distal end of the catheter lumen 924. To illustrate more clearly the construction of the valve 920, FIG. 9B shows a transverse section view along line 9B-9B of FIG. 9A, and FIG. 9C shows a transverse section view along line 9C-9C of FIG. 9A (for the sake of illustrative simplicity, the balloon 904 is not shown in FIGS. 9B-9C).

As described above with reference to the other embodiments, this catheter 900 may function as an anchored guide or track for a camera exchange (e.g., “exchange out” a duodenoscope, and “exchange in” an ultra-slim endoscope) with the valve 920 above allowing a user to remove the proximal hub (e.g., basic hub, manifold or other proximal structure that normally would preclude one from advancing/retracting a scope or other device over the proximal catheter end) without losing significant pressure from the balloon lumen 905, thereby allowing the balloon 904 to function as an anchor.

As shown in FIG. 9D, when it becomes desirable to deflate the balloon 904, a user may introduce a loop-tipped wire 998 or other flexible elongate device through the catheter lumen 924, directing it distally therethrough and allowing the ramp 997 to deflect it into the flap 996, opening the flap 996 to allow deflation of the balloon 904 and removal of the catheter 900 (e.g., in a manner similar to that shown in FIG. 1G).

FIGS. 10, 10A, and 10B show another embodiment of a catheter 1000 with a proximal actuatable valve including a hub/manifold 1010. FIG. 10 shows a partially disassembled view including a T-shaped manifold 1010, catheter body 1001, and sealing rod member 1033. Proximal and distal cannulas 1032a, 1032b are crimped or otherwise attached around the outside diameter of the catheter body 1001 in a manner that reduces the inner diameter of the catheter lumen 1024 for its length within each cannula (see FIGS. 10A-10B). The sealing rod member 1033 includes a distal sealing ball 1034, the outer surface of which is configured to frictionally sealingly contact the inner diameter of the catheter lumen 1024. The outer diameter of the distal sealing ball preferably is at least equal to or greater than the inner diameter of the cannulas 1032a, 1032b, such that the ball 1034—when disposed therebetween—cannot be moved proximally or distally past those cannulas. The proximal portion of the rod member 1033 preferably includes a grasping member 1035, shown here as a ball. The grasping member 1035 most preferably has a sufficiently low profile that the manifold 1010 can be removed proximally over it without forcing it to move longitudinally.

The sealing ball 1034 is shown as being disposed at the distal end of the rod member 1033. It should be appreciated that, in other embodiments practicable within the scope of the present invention, the rod member 1033 may extend distally beyond the sealing ball 1034 in a manner that may provide support for the catheter body 1001. The outer diameter of the body of the rod member 1033 preferably is sufficiently less than the inner diameter of the catheter lumen 1024 to permit fluid passage through the lumen when the rod body is present.

When assembled in the manner shown in FIGS. 10A-10B, compression sealing members 1043 of the manifold 1010 circumferentially, sealingly engage the catheter body 1001 and/or cannulas 1032a, 1032b with a releasable compression fit (e.g., by threaded connection). The manifold 1010 is configured with a central side branch 1018 that includes a fluid-source connector end 1012 to which an inflation fluid supply (e.g., syringe) may be attached. The distal end (not shown) of the catheter body 1001 may be configured with an inflation balloon and other features such as are shown in FIG. 4A. A branch lumen 1008 of the side branch 1018 provides a path of fluid communication to the catheter lumen 1024 via a catheter side aperture 1024a.

FIGS. 10A-10B show a partial longitudinal section view of the device 1000 of FIG. 10. FIG. 10A shows the device 1000 in an open, unsealed state where inflation fluid may freely be directed through the branch lumen 1008, catheter side aperture 1024a, and distally through the catheter lumen 1024. FIG. 10B shows the device 1000 in an actuated, sealed state. In FIG. 10B, the rod member 1033 is advanced so that the sealing ball 1034 moves distally past the catheter side aperture 1024a, creating a proximal-end seal of the catheter lumen 1024 that preferably is sufficiently strong to maintain fluid pressure within a distal anchoring balloon (not shown, see FIG. 4A and corresponding text). The distal cannula 1032b prevents the ball 1034 from moving too far distally. In preferred embodiments, a user may have a tactile sense of the ball 1034 moving distally past the catheter side aperture 1024a due to the tight tolerances of the ball's outer diameter and the inner diameter of the catheter lumen 1024.

The compression members 1043 of the manifold 1010 may be loosened, and the manifold 1010 may be removed by drawing it proximally over the proximal ends of the catheter body 1001 and rod member 1033. This removal will not disrupt the seal effected by the sealing ball 1034 with the inner diameter of the catheter lumen 1024, and will leave only the low profile/outer diameter of the catheter 1001 over which a tool or device (e.g., duodenoscope, ultra-slim intraductal endoscope, surgical device) may be advanced or withdrawn while the distal catheter end remains anchored by a balloon in the manner described above with reference to other embodiments.

In one illustrative embodiment, the catheter 1001 may be configured as a flexible catheter having an inner diameter of about 0.034 inches and an outer diameter of about 0.053 inches. The sealing ball 1034 may have an outer diameter of about 0.037 inches, such that it tightly engages and slightly compresses and/or deforms the catheter wall, providing a fluid-patent frictional sealing contact. The cannulas 1032a, 1032b preferably are rigid (e.g., metal) and may have an inner diameter of about 0.034 inches, which will not permit passage of the sealing ball 1034 therethrough. The grasping member 1035 may have an outer diameter of about 0.053 inches.

Those of skill in the art will appreciate that embodiments not expressly illustrated herein may be practiced within the scope of the present invention, including that features described herein for different embodiments may be combined with each other and/or with currently-known or future-developed technologies (including, for example, different types of valves useful for sealing a catheter lumen while allowing passage thereover of an endoscopic surgical device, or a removable low-profile clamp configured to seal the catheter lumen while allowing passage thereover of an elongate surgical device) while remaining within the scope of the claims presented here. It is therefore intended that the foregoing detailed description be regarded as illustrative rather than limiting. And, it should be understood that the following claims, including all equivalents, are intended to define the spirit and scope of this invention.

We claim:

1. An anchoring balloon catheter, comprising:
a proximal hub;
an elongate catheter body including at least one catheter inflation lumen;
an anchoring balloon disposed near a distal end of the catheter body, the balloon including a balloon lumen in fluid communication with the catheter inflation lumen and configured to be inflated in a manner engaging walls of a body lumen to inhibit longitudinal movement of the catheter body relative to the body lumen; and
an actuatable valve configured to seal a portion of the catheter inflation lumen;
wherein the hub includes a path of fluid communication with the inflation lumen and is removably attached to the catheter; and
wherein the catheter, when the valve is actuated and the hub is removed therefrom, is configured to maintain a fluid pressure in the balloon lumen and to permit passage of an elongate surgical device over a proximal end of the catheter.

2. The anchoring balloon catheter of claim 1, wherein the hub is directly attached to the catheter by a releasable fluid-tight compression seal.

3. The anchoring balloon catheter of claim 1, wherein the valve is located near the proximal end of the catheter.

4. The anchoring balloon catheter of claim 1, wherein the actuatable valve includes a plug configured to be directed distally into and sealingly occupy the proximal end of the inflation lumen.

5. The anchoring balloon catheter of claim 4, wherein the hub comprises a Tuohy-Borst seal generally longitudinally aligned with the inflation lumen and an inflation hub disposed at an angle relative to the Tuohy-Borst seal, the Tuohy-Borst seal configured for passage of a stylet configured to advance the plug distally.

6. The anchoring balloon catheter of claim 1, wherein a proximal portion of the catheter includes at least one side opening configured to permit fluid communication between the catheter inflation lumen and the hub's path of fluid communication with the inflation lumen, and wherein the actuatable valve is configured to be actuated to seal the at least one side opening.

7. The anchoring balloon catheter of claim 1, wherein the actuatable valve comprises:
an outer housing attached to the catheter body;
a piston disposed within the housing and configured to form a fluid-tight seal between the catheter body and the outer housing upon actuation of the actuatable valve; and
wherein the actuatable valve is configured such that an actuation of the valve includes moving the outer housing distally relative to the catheter body.

8. The anchoring balloon catheter of claim 7, further comprising complementary threaded surfaces on the outer housing and the catheter body, said threaded surfaces configured to facilitate moving the outer housing distally relative to the catheter body when one of the outer housing and the catheter body is rotated relative to the other.

9. The anchoring balloon catheter of claim 7, further comprising an o-ring configured to enhance a seal between at least two of the piston, the outer housing, and the catheter body.

10. The anchoring balloon catheter of claim 7, wherein the piston comprises a grooved surface configured to allow passage of a fluid between the piston and the outer housing.

11. The anchoring balloon catheter of claim 7, further comprising a first detent on one of the outer housing and the catheter body and a complementary second detent on the other of the outer housing and the catheter body, said detents configured to engage each other when the valve is in an actuated sealed state.

12. The anchoring balloon catheter of claim 1, further comprising a loop-tip disposed at the distal end of the catheter body.

13. The anchoring balloon catheter of claim 1,
wherein the actuatable valve comprises an elongate core wire extending through substantially an entire length of the catheter inflation lumen, the wire configured to occupy nearly an entire cross-sectional area of the catheter inflation lumen for at least one lengthwise portion of the catheter body;
wherein the catheter inflation lumen and the wire are complementarily configured to provide a generally fluid-tight compression seal along an inward-facing surface of the nearly-entirely occupied lengthwise portion of the catheter inflation lumen and an outward-facing surface of the wire; and
wherein the generally fluid-tight compression seal is configured to be intact below a predetermined pressure and to be overcome, permitting passage therethrough of a fluid introduced above a predetermined pressure.

14. The anchoring balloon catheter of claim 1,
wherein the actuatable valve comprises a pliable material disposed in a proximal end portion of the catheter inflation lumen, the pliable material configured to releasably seal to the hub and configured to form a seal upon contact with itself when the hub is removed, said seal sufficient to retain a fluid pressure in the balloon inflation lumen.

15. The anchoring balloon catheter of claim 1, wherein the balloon is a compliant balloon.

16. The anchoring balloon catheter of claim 1, wherein the valve is actuated by removal of the hub from the catheter body.

17. The anchoring balloon catheter of claim 1, wherein an outer diameter of the valve is not substantially greater than an outer diameter of the catheter body.

18. The anchoring balloon catheter of claim 1, wherein the valve is disposed within the balloon.

19. An anchoring balloon catheter, comprising:
a proximal hub configured for connection to an inflation source;
a distal anchor balloon including a balloon lumen and configured for anchoring within a body lumen when the balloon is inflated;
an elongate catheter body disposed between the hub and the balloon, the catheter body including at least one catheter inflation lumen configured to provide fluid communication with the balloon lumen through an aperture between the catheter lumen and the balloon lumen; and
a flap disposed over the aperture and configured to seal the when the balloon is inflated, the flap being held in a closed position by pressure of inflation fluid in the balloon lumen;
wherein the flap is configured to be opened by at least one of extension of an elongate device through the catheter lumen to contact the flap and a distal flow of inflation fluid through the catheter inflation lumen; and
wherein the proximal hub is configured to be detachable from the catheter body.

20. The catheter of claim 21, further comprising a ramped surface disposed in the catheter lumen adjacent the aperture and flap, and configured for directing a device into contact with the flap.

21. A balloon catheter, comprising:
a proximal hub configured for connection to an inflation source;
an elongate catheter body including at least one catheter inflation lumen;
an anchoring balloon disposed near a distal end of the catheter body, the balloon including a balloon lumen in fluid communication with the catheter inflation lumen and configured to be inflated in a manner engaging walls of a body lumen to inhibit longitudinal movement of the catheter body relative to the body lumen; and
a wire removably disposed through a length of the catheter inflation lumen,
the wire configured
to seal at least one portion of the catheter inflation lumen below a predetermined longitudinal pressure and
to allow passage of a fluid through the catheter inflation lumen at and above a predetermined longitudinal pressure;
wherein the hub includes a path of fluid communication with the catheter inflation lumen and is removably attached to the catheter; and
wherein the catheter, when the hub is removed therefrom, is configured to serve as a guide for an endoscopic surgical device, permitting passage of an endoscopic surgical device over a proximal catheter end.

22. A balloon catheter, comprising:
a proximal hub configured for connection to an inflation source;
an elongate catheter body;
an anchoring balloon disposed near a distal end of the catheter body, the balloon including a balloon lumen in fluid communication with the catheter inflation lumen and configured to be inflated in a manner engaging walls of a body lumen to inhibit longitudinal movement of the catheter body relative to the body lumen;
the catheter body including
a catheter inflation lumen extending through the catheter body in fluid communication with the balloon lumen; and
a proximal seal configured to releasably retain the hub portion in a proximal end of the catheter body, wherein the seal is constructed from pliable material configured as a self-sealing valve to maintain a substantially fluid tight seal of pressurized fluid within the balloon lumen when the hub is removed from the seal; and
wherein the catheter, when the hub is removed therefrom, is configured to serve as a guide for an endoscopic surgical device, permitting passage of an endoscopic surgical device over a proximal catheter end.

23. A manifold for a balloon catheter, the manifold comprising:
a first lumen at least partially transverse to a second lumen that is disposed in fluid communication with the first lumen at an intersection;
a first reduced inner diameter length of the first lumen proximal of the intersection and a second reduced inner diameter length of the first lumen distal of the intersection; and
a sealing rod disposed slidably through the first lumen, the sealing rod including a distal sealing member comprising an outer diameter that is at least equal to an inner diameter of the second reduced inner diameter length.

24. A balloon catheter system comprising:
an inflatable balloon;
an elongate tubular catheter body including a longitudinal catheter lumen disposed therethrough extending proximally from the balloon, the catheter lumen in fluid communication with the balloon;
a catheter side aperture providing a path of fluid communication with the catheter lumen;
a first reduced inner diameter length of the catheter lumen proximal of the catheter side aperture and a second reduced inner diameter length of the catheter lumen distal of the catheter side aperture;
a manifold comprising a first manifold lumen and a second manifold lumen intersecting in fluid communication with the first manifold lumen;
wherein a length of the tubular catheter body is disposed through the first manifold lumen such that the catheter side aperture is disposed in fluid communication with the second manifold lumen; and
a sealing rod disposed slidably through the catheter lumen, the sealing rod including a distal sealing member disposed between the first and second reduced inner diameter lengths of the catheter lumen and comprising an outer diameter that is at least equal to an inner diameter of the second reduced inner diameter length;
wherein a proximal end of the sealing rod is dimensioned to allow passage through the first manifold lumen such that the manifold is removable from the tubular catheter body.

25. A method for directing an elongate surgical device into a duct of a patient body, the method comprising the steps of:
providing an anchoring balloon catheter including
an elongate catheter shaft;
an anchoring balloon disposed near a distal end of the shaft;
a removable proximal hub structure and a sealing structure configured to maintain the balloon in an inflated state when the hub is removed;
navigating a distal catheter portion including the balloon into a duct of a patient;
inflating the balloon to anchor the distal catheter portion within the duct;
actuating the sealing structure; and
removing the proximal hub structure from the catheter.

26. The method of claim 25, further comprising steps of:
directing the proximal catheter end into a lumen of an elongate surgical device; and
advancing the elongate surgical device along the catheter into the duct.

27. The method of claim 26, further comprising steps of:
deflating the balloon; and
removing the catheter through the elongate surgical device.

28. The method of claim 26, wherein the elongate surgical device comprises an ultra-slim endoscope including a working channel.

29. The method of claim 28, further comprising a step of introducing a surgical tool to the common bile duct via the working channel of the ultra-slim endoscope.

30. The method of claim 29, wherein the step of navigating further comprises providing a duodenoscope, directing a distal portion of the duodenoscope adjacent the Sphincter of Oddi in a patient, cannulating the Sphincter of Oddi via sphincterotomy, and directing the balloon catheter through the cannulated sphincter into a duct.

31. The method of claim 29, wherein the sealing structure is configured as a valve disposed at a location selected from at a proximal catheter end, within the balloon, and near the proximal catheter end.

32. A method for performing a medical procedure in a patient's bile duct, the method comprising the steps of:
providing an endoscope having an accessory channel;
advancing the endoscope into a patient;
positioning a distal accessory channel opening adjacent to the patient's Sphincter of Oddi;
providing an anchoring balloon catheter including
an elongate catheter shaft;
an anchoring balloon disposed near a distal end of the shaft;
a removable proximal hub structure on a proximal catheter end;
and a sealing structure configured to maintain the balloon in an inflated state when the hub is removed;
navigating a distal catheter end portion including the balloon through the endoscope accessory channel and into a bile duct of a patient;
inflating the balloon to anchor the distal catheter end portion in a duct;
actuating the proximal sealing structure;
removing the proximal hub structure from the catheter, freeing a proximal end of the catheter; and
withdrawing the endoscope over the proximal catheter end.

33. The method of claim 32, further comprising, after the step of withdrawing the endoscope, the steps of:
directing the proximal catheter end into a working channel of an ultra-slim endoscope; and
directing the ultra-slim endoscope along the catheter into the common bile duct

34. The method of claim 33, further comprising steps of:
deflating the balloon; and
removing the catheter via a working channel of the ultra-slim endoscope.

35. The method of claim 34, further comprising a step of introducing a surgical tool to a common bile duct of the patient via the working channel of the ultra-slim endoscope.

36. The method of claim 32, wherein the step of providing an endoscope includes providing a duodenoscope, and the step of navigating includes cannulating a Sphincter of Oddi of the patient via sphincterotomy, and directing the balloon catheter through the cannulated sphincter.

37. The method of claim 32, wherein the sealing structure comprises a valve structure.

38. The method of claim 37, wherein the valve structure seals itself upon removal of the hub from the catheter.

39. The method of claim 37, wherein the valve includes a plug configured to be directed distally into and sealingly occupy the proximal end of the inflation lumen.

40. The method of claim 37, wherein the valve comprises a pliable material disposed in a proximal end portion of the catheter, the pliable material configured to releasably seal to the hub and configured to form a seal upon contact with itself when the hub is removed, said seal sufficient to retain a fluid pressure in the balloon.

41. The method of claim 37, wherein the valve is disposed within the balloon.
